(19) United States
(12) Patent Application Publication
(10) Pub. No.: US 2007/0104664 A1
(43) Pub. Date: May 10, 2007

(54) TREATMENT OF TOENAIL FUNGUS

(75) Inventors: George Maltezos, Fort Salonga, NY (US); Axel Scherer, Laguna Beach, CA (US)

Correspondence Address: DAVIS WRIGHT TREMAINE LLP, 865 FIGUEROA STREET, SUITE 2400, LOS ANGELES, CA, 90017-2566, US

(73) Assignee: CALIFORNIA INSTITUTE OF TECHNOLOGY, Pasadena, CA (US)

(21) Appl. No.: 11/552,851

(22) Filed: Oct. 25, 2006

Related U.S. Application Data
(60) Provisional application No. 60/730,545, filed on Oct. 26, 2005.

(51) Int. Cl. A61K 8/18; A61N 1/00

(52) U.S. Cl. 424/61; 607/86

(57) ABSTRACT

Aspects of the invention disclosed herein relate to devices that provide heat and/or ultraviolet light to fingernails or toenails that are afflicted by onychomycosis, as well as methods for treating onychomycosis by application of heat and/or ultraviolet light. The devices of the present invention may be used in connection with systemic and/or topical anti-fungal agents to treat onychomycosis.

### CROSS-REFERENCE TO RELATED APPLICATIONS

This application claims the benefit of priority to U.S. Provisional Application Ser. No. 60/730,545, filed Oct. 26, 2005, which is incorporated by reference herein in its entirety.

### GOVERNMENT RIGHTS

The invention described herein was made in the performance of work under a grant from the National Science Foundation (No. DMI0328162; Univ of Illinois Subcontract 2003-07452-1), and is subject to the provisions of Public Law 96-517 (35 USC 202) in which the Contractor has elected to retain title.

### FIELD OF THE INVENTION

Aspects of the present invention relate generally to the treatment of onychomycosis using heat, ultraviolet light, and combinations thereof.

### BACKGROUND

It is estimated that up to 35 million Americans have onychomycosis, a condition relating to fungal infections of the toenails or fingernails. Onychomycosis is often caused by yeast, dermatophytes, or other molds, and represents approximately 50% of all nail disorders. Toenail infection accounts for approximately 80% of onychomycosis incidence, while fingernails are affected in about 20% of the cases. Dermatophytes are the most frequent cause of nail plate invasion, particularly in toenail onychomycosis. Onychomycosis caused by a dermatophyte is termed generally termed tinea unguium. Trichophyton rubrum is by far the most frequently isolated dermatophyte, followed by T. mentagrophytes. Distal subungual onychomycosis is the most common presentation of tinea unguium, with the main site of entry through the hyponychium (the thickened epidermis underneath the free distal end of a nail) progressing in time to involve the nail bed and the nail plate. Discoloration, onycholysis, and accumulation of subungual debris and nail plate dystrophy characterize the disease. The disease adversely affects the quality of life of its victims, with subject complaints ranging from unsightly nails and discomfort with footwear, to more serious complications including secondary bacterial infections.

Many methods are known for the treatment of fungal infections, including the oral and topical use of antibiotics (e.g., nystatin and amphotericin B), imidazole anti-fungal agents such as niconazole, clotrimazole, fluconazole, econazole and sulconazole, and non-imidazole fungal agents such as the allylamine derivatives terbinafine and naftifine, and the benzylamine butenafine.

However, onychomycosis has proven to be resistant to most treatments. Nail fungal infections reside in an area difficult to access by conventional topical treatment and anti-fungal drugs cannot readily penetrate the nail plate to reach the infection sites under the nail. Therefore, onychomycosis has traditionally been treated by oral administration of anti-fungal drugs; however, clearly this is undesirable due to the potential for side effects of such drugs, in particular those caused by the more potent anti-fungal drugs such as itraconazole and ketoconazole. An alternative method of treatment of onychomycosis is by removal of the nail before treating with a topically active anti-fungal agent; such a method of treatment is equally undesirable. Systemic antimycotic agents require prolonged use and have the potential for significant side effects. Topical agents have usually been of little benefit, primarily because of poor penetration of the anti-fungal agents into and through the nail mass.

Based on the limitations of the current onychomycosis therapies, there is a pronounced need in the art for new treatments for fungal infections associated with fingernails and toenails.

### SUMMARY OF THE INVENTION

The following embodiments and aspects thereof are described and illustrated in conjunction with methods and kits are meant to be exemplary and illustrative, not limiting in scope.

In one embodiment, the invention includes a device for the treatment of onychomycosis, comprising: a treatment element selected from the group consisting of a resistive heating element, an ultraviolet light element, and combinations thereof; a power source in electrical communication with the treatment element; and a positioning element to position the treatment element over a nail affected by onychomycosis. The positioning element may be selected from the group consisting of a digit sock, a ring, a clip, an adhesively attached device, and combinations thereof. The treatment element may comprise a resistive heating element, an ultraviolet light element or a combination thereof. The ultraviolet light element may be an ultraviolet light emitting diode. The power source may be a battery or a rechargeable power unit. The device may further comprise a microprocessor. The heating element may be configured between the ultraviolet light element and the nail, and the heating element may be sufficiently thin and/or constructed of a sufficiently UV-transparent material or materials such that when the device is operated an ultraviolet light emitted by the ultraviolet light element shines through the heating element and reaches the nail.

In another embodiment, the invention includes a method of treating onychomycosis in an individual, comprising: providing an onychomycosis treatment device, comprising a treatment element, a positioning element, and a power source; positioning the onychomycosis treatment device such that the treatment element is positioned over a nail affected by onychomycosis; and operating the onychomycosis treatment device to treat the onychomycosis. The treatment element may comprise a resistive heating element, an ultraviolet light element, or both. The ultraviolet light element may be an ultraviolet light emitting diode. The method may further comprise administering an anti-fungal composition the individual. Administering the anti-fungal composition may involve administering a topical anti-fungal composition, administering a systemic anti-fungal composition, or both.

In another embodiment, the invention includes a method of treating onychomycosis in a nail of an individual, comprising: heating the nail; and exposing the nail to ultraviolet light, by way of an artificial ultraviolet light source. The method may further comprise administering an anti-fungal composition the individual. Administering the anti-fungal composition may involve administering a topical anti-fungal composition, administering a systemic anti-fungal composition, or both.

In another embodiment, the invention includes a kit for treatment of onychomycosis in a nail of a subject, comprising: a device for the treatment of onychomycosis, comprising: a treatment element selected from the group consisting of a resistive heating element, an ultraviolet light element, and combinations thereof, a power source in electrical communication with the treatment element, and a positioning element to position the treatment element over a nail affected by onychomycosis; and instructions to use the device to treat onychomycosis in the subject. The treatment element may comprise a resistive heating element, an ultraviolet light element or both. The treatment element may comprise an ultraviolet light emitting diode. The may further comprise an anti-fungal composition suitable for topical and/or systemic administration to the subject, and the instructions may further include instructions to administer the anti-fungal composition to the subject.

Other features and advantages of the invention will become apparent from the following detailed description, taken in conjunction with the accompanying figures, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 shows a sock-type onychomycosis treatment device in accordance with an embodiment of the present invention.

FIG. 2 shows a ring-type onychomycosis treatment device in accordance with an embodiment of the present invention.

FIG. 3 shows an alternative ring-type onychomycosis treatment device in accordance with an embodiment of the present invention.

FIG. 4 shows a clip-type onychomycosis treatment device in accordance with an embodiment of the present invention.

FIG. 5 shows an adhesive-type onychomycosis treatment device in accordance with an embodiment of the present invention.

FIG. 6 shows a treatment element in accordance with an embodiment of the present invention.

FIG. 7 shows an alternative treatment element in accordance with an embodiment of the present invention.

FIG. 8 shows the results of exposing a fungal culture to UV light in accordance with an embodiment of the present invention.

### DETAILED DESCRIPTION OF THE INVENTION

Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described.

Embodiments of the present invention are useful for treating onychomycosis, i.e., a disease (e.g., a fungal infection) of the nail plate on the fingers or toes, collectively termed herein as “digits”. Nail fungal disease is often caused by Epidermophyton, Microsporum, and/or Trichophyton and produces nails that are opaque, white, thickened, friable, and brittle. There are a number of conditions that facilitate fungal growth on, under, and/or around nails; particularly toenails. They include lack of ultraviolet (UV) light, decreased temperature relative to the rest of the body, and excess moisture due to wearing shoes that limit evaporation. The inventive devices and methods treat onychomycosis by decreasing or eliminating at least one of the conditions that facilitate fungal growth. Inhibition of fungal growth may generally improve the condition of the nail.

As used herein, reference to a “nail” includes reference to one, or some, or all parts of a finger or toe nail, including the nail plate (the stratum corneum unguis, which is the horny compact outer layer of the nail, i.e., visible part of the nail), the nail bed (the modified area of the epidermis beneath the nail plate, over which the nail plate slides as it grows), the cuticle (the tissue that overlaps the nail plate and rims the base of the nail), the nail folds (the skin folds that frame and support the nail on three sides), the lunula (the whitish half-moon at the base of the nail), the matrix (the hidden part of the nail under the cuticle), and the hyponychium (the thickened epidermis underneath the free distal end of a nail). Nails grow from the matrix. Nails are composed largely of keratin, a hardened protein (that is also in skin and hair). As new cells grow in the matrix, the older cells are pushed out, compacted and take on the familiar flattened, hardened form of a fingernail or toenail.

The term “treatment” as used herein encompasses any treatment of onychomycosis, and includes: preventing the disease from occurring in a subject which may be predisposed to the disease but has not yet been diagnosed as having it; inhibiting the disease, i.e., arresting its development; and/or relieving the disease, i.e., causing regression of the disease. In the context of the present invention, relieving the disease means attaining improvement in the subject's condition, including, but not limited to clinical improvement, microbiological improvement, and aesthetic improvement.

Aspects of the present invention include a treatment device that provides UV light, heat, or a combination thereof to a subject. UV light has been shown to disrupt fungal biological activity and inhibit fungal growth (Brasch J. and Menz A., Mycoses. 38:197-203, 1995; Buchnicek J., Mycopathol Mycol Appl. 50:205-15, 1973). The Example below demonstrates the effect of UV light on fungal cultures. Heat may raise the temperature of the area and promote loss of moisture, as well as create a localized “fever” to kill the heat-sensitive fungi. Treatment devices disclosed herein comprise a UV light element and/or a resistive heating element. For the purposes of this application, the portion of such a device that comprises the UV light element alone, the heating element alone, or a combination of a UV light element and resistive heating element, is referred to herein as a “treatment element”.

The resistive heating element may be any suitable heater, for example, an evaporated thin metal heater, which comprises a thin metal wire encased in a plastic “sandwich”, that when exposed to an electric current, increases in temperature. The plastic sandwich may be made of a UV-transparent material such that the UV light passes through it. Alternatively, it may comprise indium tin oxide, or some other form of transparent conductor. Other suitable heating devices include those supplied by Omega.com, which comprise silicone rubber, Kapton, and fiberglass heating elements that may be conformed to various device configurations.

The UV light element may comprise a number of types of light sources, including light emitting diodes (LED) that produce at least a small degree of light in the UV spectrum (generally about 380 nm and below). Examples of UV LEDs that may be suitable for devices disclosed herein are supplied by Sensor Electronic Technology, Inc. Such devices may comprise an aluminum gallium nitride lamp system that emits light in the range of about 250 nm to about 365 nm.

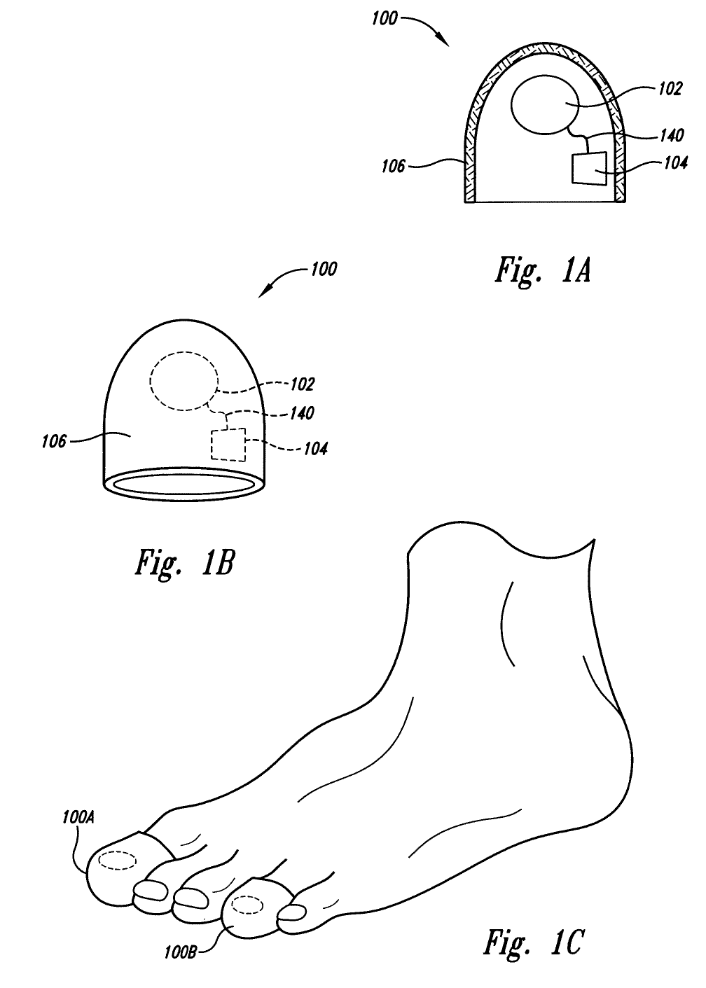

The portion of the device that positions the treatment element over the affected nail is referred to herein as the “positioning element”. A positioning element may be any physical entity that allows the treatment element to be placed on the nail of an individual such that treatment may be performed. In order to facilitate use, in a device in accordance with an embodiment of the invention, the positioning element in combination with the treatment element may be designed to suit the shape and contour of the nails of the hands and feet. FIGS. 1-5 illustrate exemplary embodiments of devices in accordance with the invention. Embodiments of positioning elements may include circular designs, ring designs, clip designs, sock designs, thimble designs, designs that wrap the whole finger, glove finger designs, designs may be linear and may be closed using an adhesive tape or hook and loop attachment (e.g., Velcro™), as well as an devices that may be directly attached to a nail with a suitable adhesive. Any other design that provides a thin and flexible device that suits the contour of the finger or toe is acceptable.

Devices in accordance with embodiments of the present invention further comprise a power source to enable the treatment element to produce heat and/or light. The power source may optionally be located in any suitable position on the treatment device. The power source may thin and flexible. The term “power source” as used herein includes, but is not limited to, any suitable cell in which chemical energy is converted to electric energy by a spontaneous electron transfer reaction. The term includes cells with non-spontaneous reactions, cells with spontaneous reactions, galvanic cells, electrolytic cells, and/or a combination thereof. Additionally, the device may be plugged into an electrical outlet to provide power, or utilize a rechargeable and/or battery-powered system. Electrical wires may provide electronic communication and/or an electrical supply among various components of the present invention.

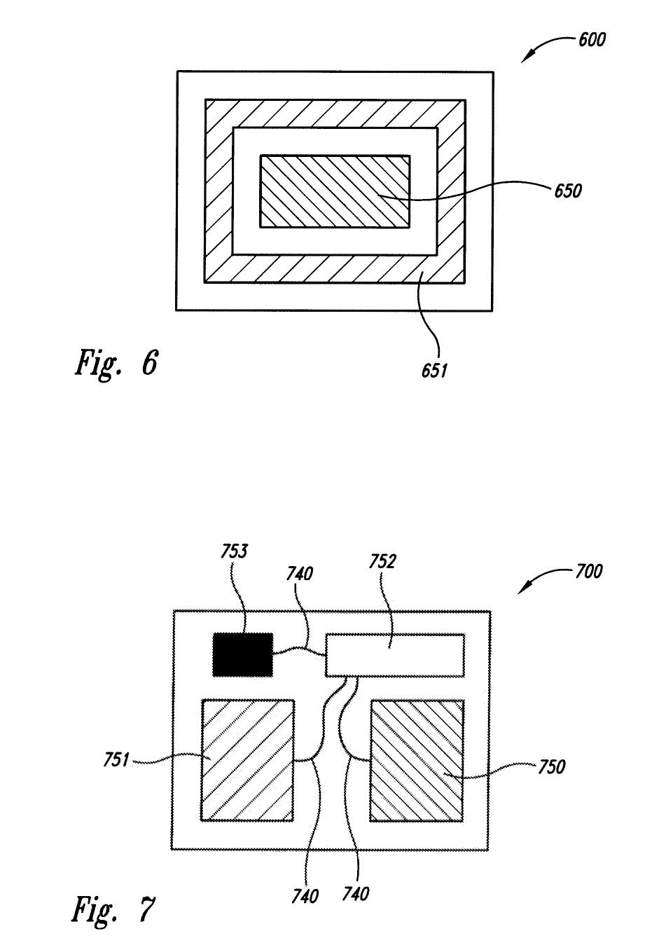

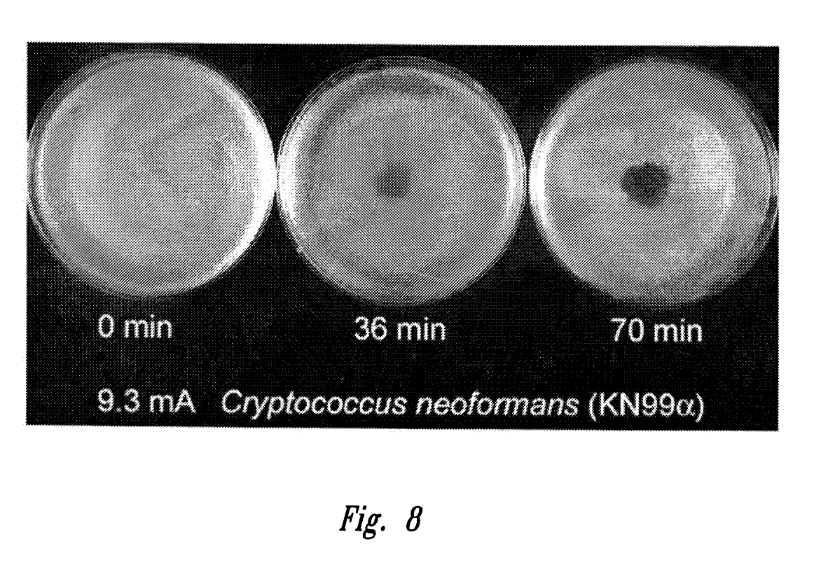

In embodiments having both an ultraviolet light element and a resistive heating element, any suitable configuration of the two elements may be used. In one configuration, a treatment element 600 may include an ultraviolet light 650 surrounded by a region of a resistive heating element 651 (FIG. 6). In another configuration, a treatment element 700 may include an ultraviolet light 750 adjacent to a resistive heating element 751 (FIG. 7). As depicted in FIG. 7, the ultraviolet light 750 and resistive heating elements 751 may be integrated such that they share a single power source, as well as circuitry or microprocessor elements 752. Alternatively, the heating element and the UV light element may be separate entities. In a still further embodiment (not shown), the heating element can be sufficiently thin and/or constructed of a sufficiently UV-transparent material or materials such that the heating element may be configured between the UV light element and the nail, and, when in operation, the UV light element may shine UV light through the heating element. It is also possible for a single power source to be connected to multiple treatment devices, so that multiple digits may be treated simultaneously.

Devices may include a circuit or microprocessor 752 that allows the user to set and/or optimize the temperature, and a temperature monitor may be used to determine the degree of heat transfer through the nail resulting from the heating element and/or the UV light element. Similarly, the device may include a UV light monitor 753 that measures the amount of UV light that passes through the nail and reaches the skin beneath, enabling the user to maintain a safe level of UV exposure. Light and heat administration may be continuous or pulsed, and may be controlled by a microprocessor 752. Pulsing of the UV light may treat the affected nail while reducing the overall UV light exposure to the patient. Electrical wiring 740 may provide electronic communication among the various elements included in a treatment element.

There are a number of different device configurations that may be used to affix a treatment element onto the nail of an individual. For example, US Patent Publication No. 2005/0038375 describes a number of different devices that may be affixed onto the toe of an individual, and is incorporated herein by reference in its entirety.

FIG. 1 depicts a flexible, wearable, sock-type embodiment 100. FIG. 1A depicts the interior of a sock-type device, and shows the approximate location of the treatment element 102. The power source 104, may be placed either inside the sock or outside, or be integrated with the treatment element, and may be in electrical communication with the treatment element 102 by virtue of wiring 140. FIG. 1B depicts the exterior of the device, and FIG. 1C depicts two sock-type devices affixed to two different toes, (100A and 100B) on the same foot. In this particular embodiment, the positioning element is the sock itself 106. The sock may comprise any suitable flexible and/or elastomeric material that is capable to conforming to a digit and positioning the treatment element over an affected nail.

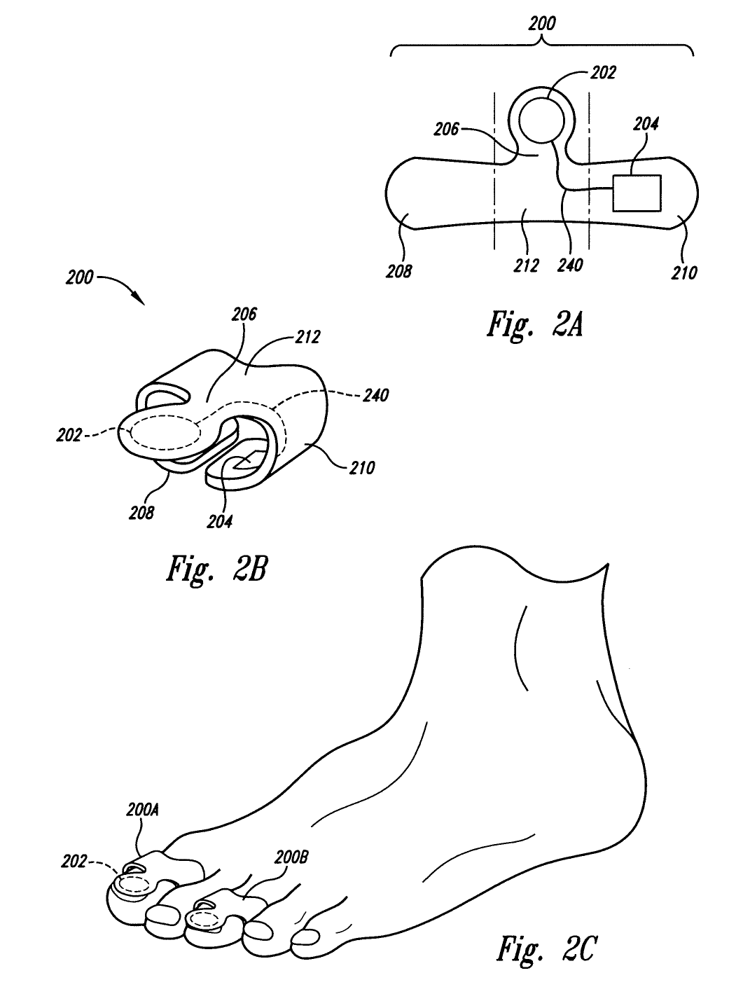

FIG. 2 depicts a ring type-embodiment 200. Ring-type embodiments may partially or completely encircle the digit to be treated by the device. FIG. 2A depicts the interior of such a device, showing the approximate placement of the treatment element 202. The power source 204 is also arbitrarily depicted on one side of the device, but may be placed in any suitable position, and may be in electrical communication with the treatment element 202 by virtue of wiring 240. The positioning element 206 comprises a right lateral portion 208, a left lateral portion 210, and a midline portion 212. The midline portion projects, in only one direction, from an imaginary line joining the left 210 and right 208 lateral portions, and is generally perpendicular to the left and right lateral portions. The treatment element 202 may be attached to the midline portion 212 such that the treatment element 202 is positioned properly over a nail. The right lateral portion 208 and the left lateral portion 210 may be made from a flexible or deformable material that allow the device to be wrapped around a digit and conformed to the shape thereof, and may be disposed on any suitable portion of the digit. It is possible that the right lateral portion 208 and left lateral portion 210 overlap on the side of the device opposite to the midline portion 212. An adhesive may be used to attach the device to the skin of the wearer. FIG. 2B shows the exterior of the device. FIG. 2C shows two such devices 200A and 200B affixed to two different toes on the same foot.

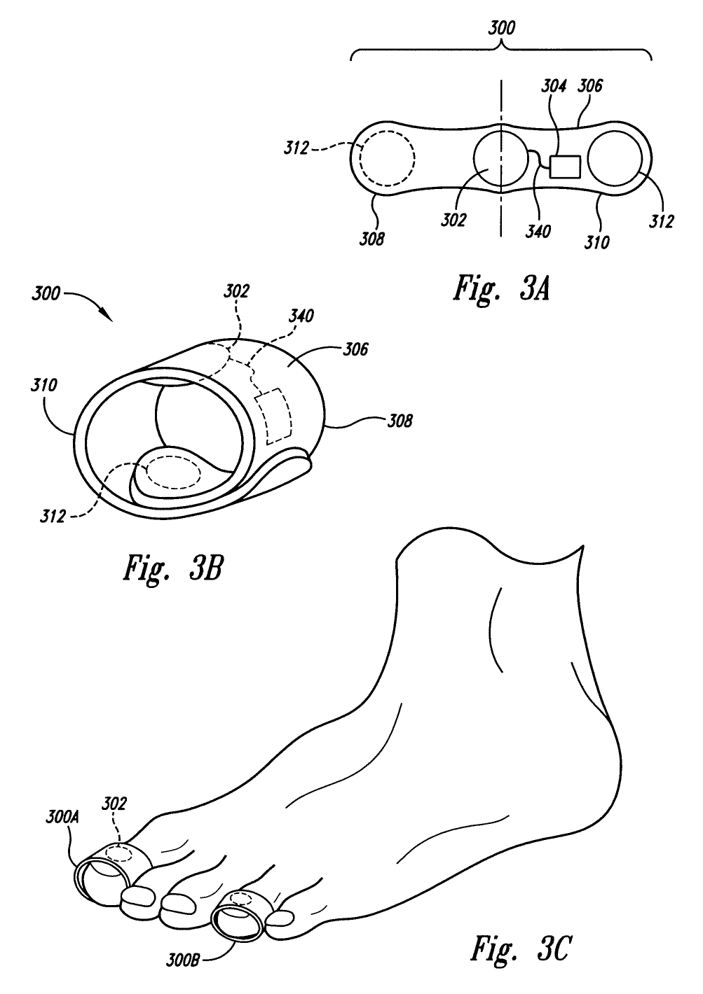

FIG. 3 shows an alternative ring-type embodiment 300. FIG. 3A depicts the interior of such a device, and shows the approximate position of the treatment element 302. The power source 304 is also depicted, and may be place in any suitable position on or near the device, and may be in electrical communication with the treatment element 302 by virtue of wiring 340. The positioning element 306 comprises a right lateral portion 308 and left lateral portion 310. As with the previous embodiment, the positioning element may partially or completely encircle the digit of the wearer. FIG. 3B depicts the device 300 with the right lateral portion 308 in contact with left lateral portion 310, creating an overlap portion 312. The overlap portion 312 may be joined by an adhesive, or alternatively, a releasable attachment material such as a hook-and-loop type material. The joined overlap portion serves to firmly affix the device on the digit of the wearer. FIG. 3C shows two such devices 300A and 300B affixed to two different toes on the same foot. In an alternate embodiment, the ring-type structure may be unitary, such that a user may slide it on a digit as with a typical ring.

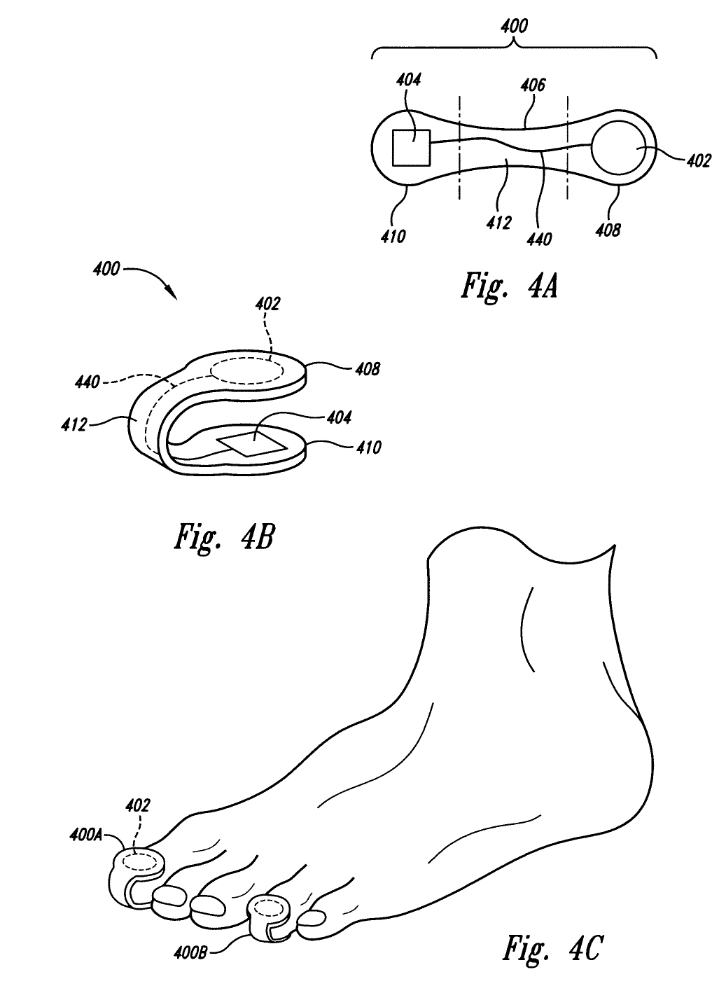

FIG. 4 depicts a clip-type, or C-shaped embodiment 400. FIG. 4A depicts an interior view of the device 500, showing the approximate placement of the treatment element 402 and power source 404. The power source 404 may be placed in any suitable position on or near the device, and may be in electrical communication with the treatment element 402 by virtue of wiring 440. The positioning element 406 may include a top portion 408, a bottom portion 410, and an intervening portion 412. The treatment element 402 may be affixed to the top portion 408. The intervening portion 412 acts to position the top portion 408 and bottom portion 410 on opposite sides of the digit and allows the treatment element 402 to be positioned over the nail. In FIG. 4B, the top 408 and bottom 410 portions are curvilinearly folded into a C-shape configuration. Any number of resilient materials may be incorporated into this embodiment of the present invention such that the C-shaped embodiment may retain its physical shape. The C-shaped conformation is suitable for use on any digit because the top 408, bottom 410, and intervening 412 portions may act together to embrace the digit. FIG. 4C shows two such devices 400A and 400B affixed to two different toes on the same foot.

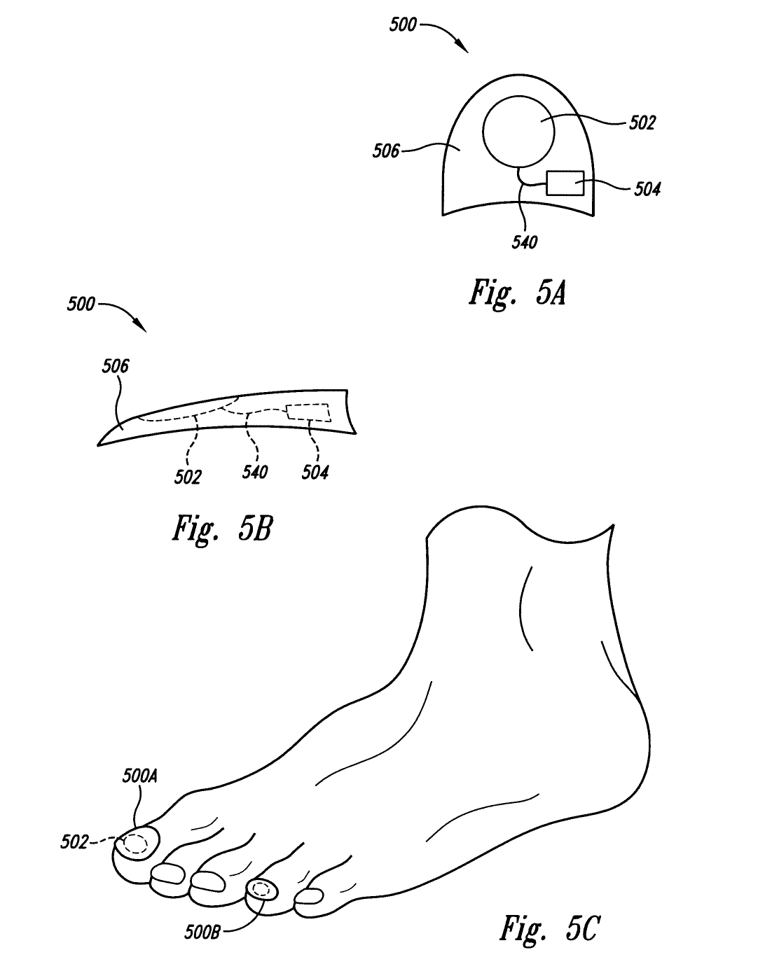

FIG. 5 depicts an embodiment of the device 500 that is designed to be directly attached to a nail by an adhesive. The adhesive may be pre-affixed to the device, or it may be separately applied to the device and/or the digit/nail by a user. FIG. 5A depicts the interior of the device 500 which comprises a treatment element 502, a power source 504, and a positioning element 506. The positioning element 506 is designed to approximate the size and shape of a fingernail or toenail, and may be affixed directly to the nail by any suitable adhesive. The power supply 504 may be positioned in any suitable place on the device, and may be in electrical communication with the treatment element 502 by virtue of wiring 540. FIG. 5B shows a side-view of the device 500. FIG. 5C depicts two such devices (500A and 500B) affixed to two different toenails on the same foot. The devices depicted in FIGS. 1-5 may be designed such that they may be worn underneath a sock and/or inside a shoe.

Another embodiment may be applied when the onychomycosis has progressed such that the nail has separated from the nail bed, leaving a space therebetween. In such a situation, the space may decrease the efficiency of treatment due to less thermal conductivity through the space. In this situation, a thermocouple may be placed underneath the nail and the current may be set to produce the most efficacious temperature at the nail bed without causing discomfort to the wearer. Optionally, a UV light may then also be applied to the external surface of the nail.

Depending on the sensitivity of the particular fungus, it may be necessary to separate the light and the heat treatment. In some cases, the patient may be treated with a “shock” treatment of UV light and heat in a medical office, and be told to wear a resistive heating element all day, or all night. The device used in a medical setting may be a scaled up version of the portable devices disclosed above, and may also shield the exposed skin to protect it from the UV light as well as measure the amount of UV light that passes through the nail.

Further embodiments of the invention relate to the use of UV light/heat therapy in conjunction with a medicinal anti-fungal treatment. In connection with this embodiment of the invention, treatment may include topical and/or systemic administration of a composition comprising an anti-fungal agent, in combination with the use of an electrically operated device to produce heat and/or UV light.

Treatment with the inventive device and the medicinal treatment may be administered at times and at periods independently selected from once or multiple times, and may be administered together or separately. Each administration of the treatment with the inventive device can be for a period of time lasting from several minutes to several hours.

Broadly, the anti-fungal agent may comprise an anti-fungal compound, or a pharmaceutically acceptable salt or derivative thereof. A single anti-fungal agent or mixture of such agents can be used and will be termed “anti-fungal agent” or “anti-fungal compound” herein, interchangeably. There is no particular limitation on the anti-fungal agents used in the compositions of this invention. By way of example, preferred suitable anti-fungal agents may be comprised of polyenes, e.g., Natamycin, Nystatin; allylamines, e.g., Naftifine, Terbinafine; imidazoles, e.g., Bifonazole, Chlotrimazole, Econazole, Fenticonazole, Ketocanazole, Miconazole, Oxiconazole; triazoles, e.g., Fluconazole, Itraconazole, Terconazole, tolnaftate, ciclopirox, undecylenic acid, sulbentine; and morpholines, e.g., amorolfine, and the related morpholines. The amount of the anti-fungal agent present in the composition, as described hereinbelow, may be an amount that is therapeutically effective, i.e. the amount that will result in an effective treatment of the onychomycosis when applied in accordance with the instructions described herein. The anti-fungal agent may be administered topically, systemically or a combination thereof, in accordance with alternate embodiments of the present invention. A combination of various anti-fungal agents may be administrered; for instance, one anti-fungal agent that is administered topically, and another that is administered systemically. The administration of multiple anti-fungal agents can be administered simultaneously, separately, and in independently varying dosages, based on clinical considerations well known to those of skill in the art. Indeed, the amount of the anti-fungal agent or agents administered in accordance with various embodiments of the invention will vary depending upon a variety of factors, including but not limited to the characteristics of the anti-fungal agent (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the composition, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

In accordance with another embodiment of the present invention, treatment may be accomplished with the use of a kit. The kits are useful for practicing the inventive method of treating onychomycosis. The kit is an assemblage of materials or components, including at least the inventive device. In some embodiments, the kit also contains at least one anti-fungal agent suitable for topical or systemic administration.

Instructions for use may be included in the kit. “Instructions for use” typically include a tangible expression describing the technique to be employed in using the components of the kit to effect a desired outcome, such as to treat onychomycosis. For example, the instructions may comprise instructions to administer heat and/or UV light to a subject with an inventive device, and may, optionally, also include instructions to administer one or more anti-fungal agents to the subject.

Optionally, the kit also contains other useful components, such as, diluents, buffers, pharmaceutically acceptable carriers, syringes, catheters, applicators, pipetting or measuring tools, or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of an inventive composition containing, for example, an anti-fungal agent as described above. The packaging material generally has an external label which indicates the contents and/or purpose of the kit and/or its components.

The following example is provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### EXAMPLE

FIG. 6 shows the effect of UV light on cultures of Cryptococcus neoformans. Plate cultures of Cryptococcus neoformans were exposed to UV light for 0, 36, or 70 minutes.

While the description above refers to particular embodiments of the present invention, it will be understood that many modifications may be made without departing from the spirit thereof. The presently disclosed embodiments are therefore to be considered in all respects as illustrative and not restrictive. All publications, patents, and patent applications cited herein are hereby incorporated by reference in their entirety for all purposes.

1. A device for the treatment of onychomycosis, comprising:
a treatment element selected from the group consisting of a resistive heating element, an ultraviolet light element, and combinations thereof;
a power source in electrical communication with the treatment element; and
a positioning element to position the treatment element over a nail affected by onychomycosis.

2. The device of claim 1, wherein the positioning element is selected from the group consisting of a digit sock, a ring, a clip, an adhesively attached device, and combinations thereof.

3. The device of claim 1, wherein the positioning element comprises a digit sock.

4. The device of claim 1, wherein the positioning element comprises a ring.

5. The device of claim 1, wherein the positioning element comprises a clip.

6. The device of claim 1, wherein the positioning element comprises an adhesively-attached device.

7. The device of claim 1, wherein the treatment element comprises a resistive heating element.

8. The device of claim 1, wherein the treatment element comprises an ultraviolet light element.

9. The device of claim 1, wherein the treatment element comprises an ultraviolet light emitting diode.

10. The device of claim 1, wherein the treatment element comprises a resistive heating element and an ultraviolet light element.

11. The device of claim 10, wherein the ultraviolet light element is an ultraviolet light emitting diode.

12. The device of claim 1, wherein the power source is a battery.

13. The device of claim 1, wherein the power source is a rechargeable power unit.

14. The device of claim 1, further comprising a microprocessor.

15. The device of claim 1, wherein the heating element is configured between the ultraviolet light element and the nail, and the heating element is sufficiently thin and/or constructed of a sufficiently UV-transparent material or materials such that when the device is operated an ultraviolet light emitted by the ultraviolet light element shines through the heating element and reaches the nail.

16. A method of treating onychomycosis in an individual, comprising:
providing an onychomycosis treatment device, comprising a treatment element, a positioning element, and a power source;
positioning the onychomycosis treatment device such that the treatment element is positioned over a nail affected by onychomycosis; and
operating the onychomycosis treatment device to treat the onychomycosis.

17. The method of claim 16, wherein the treatment element comprises a resistive heating element.

18. The method of claim 16, wherein the treatment element comprises an ultraviolet light element.

19. The device of claim 13, wherein the treatment element comprises an ultraviolet light emitting diode.

20. The method of claim 16, further comprising administering an anti-fungal composition to the individual.

21. The method of claim 20, wherein administering an anti-fungal composition further comprises administering a topical anti-fungal composition, administering a systemic anti-fungal composition, or both.

22. A method of treating onychomycosis in a nail of an individual, comprising:
heating the nail; and
exposing the nail to ultraviolet light, by way of an artificial ultraviolet light source.

23. The method of claim 22, further comprising administering an anti-fungal composition to the individual.

24. The method of claim 23, wherein administering an anti-fungal composition further comprises administering a topical anti-fungal composition, administering a systemic anti-fungal composition, or both.

25. A kit for treatment of onychomycosis in a nail of a subject, comprising:
a device for the treatment of onychomycosis, comprising:
a treatment element selected from the group consisting of a resistive heating element, an ultraviolet light element, and combinations thereof,
a power source in electrical communication with the treatment element, and
a positioning element to position the treatment element over a nail affected by onychomycosis; and
instructions to use the device to treat onychomycosis in the subject.

26. The kit of claim 25, wherein the treatment element comprises a resistive heating element.

27. The kit of claim 25, wherein the treatment element comprises an ultraviolet light element.

28. The kit of claim 25, wherein the treatment element comprises an ultraviolet light emitting diode.

29. The kit of claim 25, wherein the treatment element comprises a resistive heating element and an ultraviolet light element.

30. The kit of claim 25, further comprising an anti-fungal composition suitable for topical and/or systemic administration to the subject, wherein the instructions further include instructions to administer the anti-fungal composition to the subject.
